(19) United States
(12) Patent Application Publication
(10) Pub. No.: US 2021/0038545 A1
(43) Pub. Date: Feb. 11, 2021

(54) USE OF SHORT-CHAIN FATTY ACIDS FOR THE TREATMENT OF BACTERIAL SUPERINFECTIONS POST-INFLUENZA

(71) Applicants: NSERM (INSTITUT NATIONAL DE LA SANTE ET DE LA RECHERCHE MÉDICALE), Paris (FR); INSTITUT PASTEUR DE LILLE, Lille (FR); CENTRE NATIONAL DE LA RECHERCHE SCIENTIFIQUE (CNRS), Paris (FR); UNIVERSIDADE FEDERAL DE MINAS GERAIS - UFMG, Belo Horizonte, Pampulha (BR); UNIVERSITÉ DE LILLE, Lille (FR)

(72) Inventors: François TROTTEIN, Lille Cedex (FR); Adeline BARTHELEMY, Lille Cedex (US); Valentin SENCIO, Lille Cedex (FR); Mauro Martins TEIXEIRA, Lille Cedex (FR); Angelica THOMAZ VIEIRA, Lille Cedex (FR); Luciana TAVARES, Lille Cedex (FR)

(21) Appl. No.: 16/965,663

(22) PCT Filed: Jan. 30, 2019
(86) PCT No.: PCT/EP2019/052194

§ 371 Date: Jul. 29, 2020

(30) Foreign Application Priority Data
Jan. 31, 2018 (EP) 18305095.4

(51) Int. Cl. A61K 31/19; A61P 31/04; G01N 33/92

(52) U.S. Cl. CPC A61K 31/19; A61B 10/0038; G01N 33/92; A61P 31/04

(57) ABSTRACT

Severe influenza is associated with defects in pulmonary innate immunity, a phenomenon leading to secondary bacterial infections. The gut microbiota can control immune/inflammatory responses locally and at distant sites. The inventors hypothesized that perturbation of the gut microbiota during severe influenza might participate in bacterial superinfection post-influenza. Their data demonstrated that influenza infection profoundly altered the functionality of the gut microbiota as assessed by the altered production of short chain fatty acids (SCFAs). Remarkably, treatment of colonized (IAV microbiota) mice or IAV-infected mice with acetate, the main SCFA found systematically, reinforced host defenses against S. pneumoniae. The present invention thus relates to the use of short-chain fatty acids for the treatment of bacterial superinfections post-influenza.

### FIELD OF THE INVENTION

The present invention relates to methods for the treatment of bacterial superinfections post-influenza.

### BACKGROUND OF THE INVENTION

Despite vaccination programs and antiviral drugs, influenza A virus (IAV) infection is responsible for widespread morbidity and mortality every winter (Monto, 2008; Taubenberger et Morens, 2008). Influenza infections can also result in sporadic pandemics that can be devastating; the 1918 pandemic led to the death of 50 million people. Severe bacterial (e.g. pneumococcal) infections are commonly associated with influenza and are significant contributors to the excess morbidity and mortality of influenza (Morens et al., 2008; Brundage, 2006). Among mechanisms leading to enhanced susceptibility to bacterial infection, disruption of pulmonary defenses plays a critical role. Numerous reports have demonstrated that the inability to control bacterial outgrowth is associated with impaired functions of numerous sentinel and effector immune cells including dendritic cells, macrophages, and neutrophils (Balinder et Standiford, 2010; van der Sluijs et al., 2010; McCullers, 2006; Snelgrove et al., 2011; Short et al., 2014; Barthelemey et al., 2016). In the current study, we hypothesized that gut disorders during influenza might play a critical role in disrupted pulmonary defenses and secondary bacterial infection.

The gastrointestinal tract hosts a complex ecosystem with enormous microbial diversity. This regulated, finely balanced interplay enables the establishment and persistence of local and systemic immune homeostasis (for reviews, Clarke, 2014; Clemente et al., 2014; Thaiss et al., 2016). The impact of commensal microbes on host immune responses is not limited to the gut compartment (barrier function and gut homeostasis) but extends to systemic compartments and distant mucosal interfaces including the lungs (Marshland et Gollwitzer, 2014). The mechanism by which the bacterial flora educates the immune system and regulates the size and/or the functions of the steady-state immune cell pool depends on microbial components (e.g. pathogen-associated molecular patterns) and metabolites and their interactions with intestinal cells and bone marrow-resident progenitor cells (Hooper et al., 2012; Belkaid et Hand, 2014). Recent evidence demonstrate that healthy microbiota has a critical role in host defense during respiratory bacterial infections, including Streptococcus pneumoniae (Clarke, 2014; Schuijt et al., 2016; Gray et al., 2017). In this setting, it appears that the gut microbiota calibrates the responsiveness of the lung's antibacterial immunity by providing signals that shape the functions of effector immune cells—including (alveolar) macrophages and/or neutrophils (Schuijt et al., 2016). In another recent study, Gray and colleagues demonstrated that signals emanating from the gut microbiota act on the recruitment of IL-22-producing ILC3 in the lungs and that this process is essential to control S. pneumoniae (Gray et al., 2017).

Pathological situations including infectious and chronic inflammatory and metabolic disorders can modify the diversity and composition of the gut microbiota; this process is referred to as dysbiosis (Hooper et al., 2012; Maslowski et al., 2009). There is a strong relationship between specific variations in the intestinal microbiota and disease susceptibility, although clear causal relationships are still speculative (Arpaia et al., 2013). Changes in intestinal bacterial communities can in turn exacerbate disease outcomes, as demonstrated by transfer experiments with dysbiotic microbiota (Levy et al., 2017). Emerging evidences indicate that acute respiratory infections, including severe influenza, can lead to alteration of the gut microbiota (Wang et al., 2014; Lu e al., 2014; Qin et al., 2015; Deriu et al., 2016; Bartley et al., 2017). However, influenza-associated “dysbiosis” remains to be fully characterized and explored from a functional point of view. Moreover, whether changes in the composition of the gut microbiota during influenza infection affect at distance respiratory bacterial infections is yet to be defined.

Short-chain fatty acids (SCFAs) represent the major metabolites of the gut microbiota (Smith et al., 2013; Wong et al., 2006). These end-products of fermentation of macronutrients are highly concentrated in the gut lumen and can distribute systemically via the blood. Emerging evidence suggest that SCFAs act locally and at distant sites to exert many physiological functions through a variety of mechanisms (Maslowski et al., 2009; Canani et al., 2001; Tan et al., 2014; Huflhagle, 2014; Macia et al., 2015). Among them, SCFAs activate free fatty acid receptors (FFAR) including FFAR2 (also known as GPR43) and FFAR3 (also known as (GPR41) (Milligan et al., 2017). The potential role of SCFAs in host defenses against pulmonary infections is still elusive. Several factors, including dietary substances, can affect the concentration of SCFAs, including acetate (the predominant member), propionate and butyrate (Topping et Clifton, 2001; Macfarlane et Macfarlane, 2003; Yang et al., 2013; Tan et al., 2016). However the role of SCFAs in the treatment of bacterial superinfections post-influenza has never been investigated.

### SUMMARY OF THE INVENTION

The present invention relates to methods for the treatment of bacterial superinfections post-influenza. In particular, the present invention is defined by the claims.

### DETAILED DESCRIPTION OF THE INVENTION

The inventors first investigated the role of short-chain fatty acids (SCFAs) in host defense against Streptococcus pneumoniae, the leading cause of bacterial pneumonia worldwide. They also hypothesize that perturbation of microbiota composition/activity during severe influenza could impact on the production of SCFAs, thus influencing lung immunity. The inventors show that preventive SCFA (acetate) treatment can enhance lung defenses against pneumococcal infection. The protective effect is dependent on FFAR2. During severe influenza infection (H3N2 and H1N1), there is a profound decreased production of SCFAs by the gut microbiota, particularly 7 days post-infection (p.i.), which is the peak of susceptibility to secondary bacterial infection. Remarkably, fecal transfer experiments demonstrate that influenza-experienced microbiota enhances susceptibility to pneumococcal infection in recipient mice. Finally, the inventors report that supplementation with short-chain fatty acid (e.g. acetate) during influenza enhances pulmonary immune defenses against secondary S. pneumoniae infection. This treatment reduces local bacterial outgrowth and dissemination and enhances the survival rate of double-infected mice. Together, drop in SCFA production during severe influenza contributes to impaired host defense against secondary bacterial infection. The finding might help to define predictive markers of bacterial (super)infection and/or to develop therapeutic approaches to combat them.

Accordingly, the first object of the present invention relates to a method of treating a bacterial superinfection post-influenza in a subject in need thereof comprising administering to the subject a therapeutically effective amount of at least one short-chain fatty acid (SCFA). In some embodiment, the SCFA is selected from free fatty acid receptor 2 (FFAR2) agonist or free fatty acid receptor 3 (FFAR3) agonist.

According to the invention the subject suffers or has suffered from an influenza infection. As used herein, the term “influenza infection” has its general meaning in the art and refers to the disease caused by an infection with an influenza virus. In some embodiments of the invention, influenza infection is associated with Influenza virus A or B. In some embodiments of the invention, influenza infection is associated with Influenza virus A. In some specific embodiments of the invention, influenza infection is cause by influenza virus A that is H1N1, H2N2, H3N2 or H5N1.

As used herein, the term “bacterial superinfection post-influenza” has its general meaning in the art and refers to a bacterial infection (e.g. bacterial pneumonia) which occurs in a subject who suffers or has suffered from an influenza infection. Typically, the bacterial superinfection occurs within 4, 5, 6, 7, 8, 9, 10, 11, 12, 13, 14, 15, 16, 17, 18, 19, 20, 21, 22, 23, 24, or 25 days after influenza infection. The method of the present invention is particularly suitable for the treatment of a bacterial superinfection post-influenza such as, but not limited to infections of the lower respiratory tract (e.g., pneumonia), middle ear infections (e.g., otitis media) and bacterial sinusitis. The bacterial superinfection may be caused by numerous bacterial pathogens. For example, they may be mediated by at least one organism selected from the group consisting of: Streptococcus pneumoniae; Staphylococcus aureus; Haemophilus influenza, Myoplasma species and Moraxella catarrhalis.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The method of the present invention is particularly suitable for subjects who are identified as at high risk for developing a bacterial superinfection post-influenza, including subjects who are at least 50 years old, subjects who reside in chronic care facilities, subjects who have chronic disorders of the pulmonary or cardiovascular system, subjects who required regular medical follow-up or hospitalization during the preceding year because of chronic metabolic diseases (including diabetes mellitus), renal dysfunction, hemoglobinopathies, or immunosuppression (including immunosuppression caused by medications or by human immunodeficiency [HIV] virus); children less than 14 years of age, patients between 6 months and 18 years of age who are receiving long-term aspirin therapy, and women who will be in the second or third trimester of pregnancy during the influenza season. More specifically, it is contemplated that the method of the invention is suitable for the treatment of bacterial superinfection post-influenza in subjects older than 1 year old and less than 14 years old (i.e., children); subjects between the ages of 50 and 65, and adults who are older than 65 years of age.

As used herein, the term “short-chain fatty acid” or “SCFA” has its general meaning in the art and refers to aliphatic carboxylic acids composed of 1 to 6 carbon atoms, which may be linear or branched. Suitable short-chain fatty acids include: formic acid; acetic acid; propionic acid; butyric (butanoic) acid; isobutyric (2-methylbutanoic) acid; valeric (pentanoic) acid; isovaleric (3-methylbutanoic); and caproic (hexanoic) acid and analogues including halogenated derivatives, such as dichloroacetate (DCA). SCFA are highly abundant in the colon but can also be detected in the blood. In some embodiments, the SCFA is selected from saturated fatty acids comprising six or less carbon atoms, or 5 or less carbon atoms. In some embodiments, the SCFA is formic acid, acetic acid, propionic acid, butyric acid, isobutyric acid, valeric acid, isovaleric acid or caproic acid, preferably acetic acid, propionic acid and butyric acid. In some embodiment, the SCFA is also a salt or an ester selected from formate, acetate, propionate, butyrate, isobutyrate, valerate, isovalerate, caproate, preferably acetate, propionate or butyrate.

As used herein, the term “free fatty acid receptor” or “FFAR” has its general meaning in the art and refers to a G-protein coupled receptors which binds free fatty acids. FFAR includes 4 receptors: FFAR1, FFAR2, FFAR3 and FFRA4. In some embodiment, the FFAR is selected from free fatty acid receptor 2 (FFAR2) or free fatty acid receptor 3 (FFAR3).

As used herein, the term “FFAR2 agonist” or “FFAR3 agonist” refers to an agonist of FFAR2 or FFAR3. As used herein, the term “agonist” has its general meaning in the art and refers to a substance that binds to a receptor and activates the receptor to produce a biological response.

In some embodiments, the SCFA is administered to the subject in the form of a nutritional composition. As used herein, the term “nutritional composition” means a composition which nourishes a subject. This nutritional composition is usually to be taken enterally, orally, parenterally or intravenously, and it usually includes a lipid or fat source and optionally a protein source and/or optionally a carbohydrate source and/or optionally minerals and vitamins. Preferably, the nutritional composition is for oral use and thus represents a food composition.

In some embodiments, the food composition is selected from complete food compositions, food supplements, nutraceutical compositions, and the like. The composition of the present invention may be used as a food ingredient and/or feed ingredient. The food ingredient may be in the form of a solution or as a solid—depending on the use and/or the mode of application and/or the mode of administration. As used herein, the term “food” refers to liquid (i.e. drink), solid or semi-solid dietetic compositions, especially total food compositions (food-replacement), which do not require additional nutrient intake or food supplement compositions. Food supplement compositions do not completely replace nutrient intake by other means. As used herein the term “food ingredient” or “feed ingredient” includes a formulation which is or can be added to functional foods or foodstuffs as a nutritional supplement. By “nutritional food” or “nutraceutical” or “functional” food, is meant a foodstuff which contains ingredients having beneficial effects for health or capable of improving physiological functions. By “food supplement”, is meant a foodstuff having the purpose of completing normal food diet. A food supplement is a concentrated source of nutrients or other substances having a nutritional or physiological effect, when they are taken alone or as a combination in small amounts. According to the invention, “functional food” summarizes foodstuff and corresponding products lately developed to which importance is attributed not only due to them being valuable as to nutrition and taste but due to particular ingredient substances. According to the invention, the middle- or long-term maintenance and promotion of health are of importance. In this context, non-therapeutic uses are preferred. The terms “nutriceuticals”, “foodsceuticals” and “designer foods”, which also represent embodiments of the invention, are used as synonyms, partly, however, also in a differentiated way. The preventive aspect and the promotion of health as well as the food character of the products are, however, best made clear by the term functional food. In many cases, these relate to products accumulated by assortment and selection (as is also the case in the present invention), purification, concentration, increasingly also by addition. Isolated effective substances, in particular in form of tablets or pills, are not included. Accordingly, functional foods are ordinary foods that have components or ingredients (such as those described herein) incorporated into them that impart to the food a specific functional e.g. medical or physiological benefit other than a purely nutritional effect.

In some embodiments, the composition typically comprises carriers or vehicles. “Carriers” or “vehicles” mean materials suitable for administration and include any such material known in the art such as, for example, any liquid, gel, solvent, liquid diluent, solubilizer, or the like, which is non-toxic and which does not interact with any components of the composition in a deleterious manner. Examples of nutritionally acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

In some embodiments, the composition comprises any other ingredients or excipients known to be employed in the type of composition in question. Non limiting examples of such ingredients include: proteins, amino acids, carbohydrates, oligosaccharides, lipids, prebiotics or probiotics, nucleotides, nucleosides, other vitamins, minerals and other micronutrients.

In some embodiments, the composition may comprise one or more protein. As used herein, the term “protein” refers to both proteins derived from a source of protein, to peptides and to free amino acids in general. There can be one or several proteins. The type of protein is not believed to be critical to the present invention provided that the minimum requirements for SCFA content are met. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions. The proteins can be at least partially hydrolyzed in order to enhancement of oral tolerance to allergens, especially food allergens. In that case the composition is a hypoallergenic composition.

In some embodiments, the composition contains a carbohydrate source, preferably as prebiotics, or in addition to prebiotics. Any carbohydrate source conventionally found in infant formulae such as lactose, saccharose, maltodextrin, starch and mixtures thereof may be used although the preferred source of carbohydrates is lactose.

In some embodiments, the composition comprises a probiotic. As used herein the term “probiotic” is meant to designate live microorganisms which, they are integrated in a sufficient amount, exert a positive effect on health, comfort and wellness beyond traditional nutritional effects. Probiotic microorganisms have been defined as “Live microorganisms which when administered in adequate amounts confer a health benefit on the host” (FAO/WHO 2001). Non limiting examples of probiotics include: Bifidobacterium, Lactobacillus, Lactococcus, Enterococcus, Streptococcus, Kluyveromyces, Saccharoymces, Candida, in particular selected from the group consisting of Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof, preferably selected from the group consisting of Bifidobacterium longum NCC3001 (ATCC BAA-999), Bifidobacterium longum NCC2705 (CNCM 1-2618), Bifidobacterium longum NCC490 (CNCM 1-2170), Bifidobacterium lactis NCC2818 (CNCM I-3446), Bifidobacterium breve strain A, Lactobacillus paracasei NCC2461 (CNCM 1-2116), Lactobacillus johnsonii NCC533 (CNCM 1-1225), Lactobacillus rhamnosus GG (ATCC53103), Lactobacillus rhamnosus NCC4007 (CGMCC 1.3724), Enterococcus faecium SF 68 (NCC2768; NCIMB10415), and combinations thereof. In an embodiment of the invention, the infant formula further includes a probiotic strain such as a probiotic bacterial strain in an amount of from 10⁶ to 10¹¹ cfu/g of composition (dry weight).

In some embodiments, the composition comprises one or more prebiotic. None limiting examples of prebiotics include: oligosaccharides optionally containing fructose, galactose, mannose; dietary fibers, in particular soluble fibers, soy fibers; inulin; and combinations thereof. Preferred prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof, and combinations of the foregoing.

In one embodiment, the composition comprises one or more vitamin. Vitamins may be folic acid, vitamin B12 and vitamin B6, in particular folic acid and vitamin B12, in particular folic acid. In some embodiments, the composition comprises one or more vitamin which is lipid-soluble, for example one or more of vitamin A, vitamin D, vitamin E and vitamin K.

In some embodiments, the composition further comprises one or more mineral. Examples of minerals are sodium, potassium, chloride, calcium, phosphate, magnesium, iron, zinc, copper, selenium, manganese, fluoride, iodine, chromium, or molybdenum. The minerals are usually added in salt form. The minerals may be added alone or in combination.

In some embodiments, the composition contains emulsifiers. Examples of food grade emulsifiers typically include diacetyl tartaric acid esters of mono- and di-glycerides, lecithin and mono- and di-glycerides. Similarly suitable salts and stabilisers may be included.

In some embodiments, the composition contains protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition may also contain conventional additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. In all cases, such further components will be selected having regard to their suitability for the intended recipient.

In some embodiments, the composition is a fermented dairy product or dairy-based product, which is preferably administered or ingested orally one or more times daily. Fermented dairy products include milk-based products, such as (but not limited to) deserts, yoghurt, yoghurt drinks, quark, kefir, fermented milk-based drinks, buttermilk, cheeses, dressings, low fat spreads, fresh cheese, soy-based drinks, ice cream, etc. Alternatively, in some embodiments, food and/or food supplement compositions may be non-dairy or dairy non fermented products (e.g. strains or cell-free medium in non-fermented milk or in another food medium). In some embodiments, the SCFA is dispersed in a food (e.g. in milk) or non-food medium. Non-fermented dairy products may include ice cream, nutritional bars and dressings, and the like. Non-dairy products may include powdered beverages and nutritional bars, and the like. The products may be made using known methods, such as adding an effective amount of SCFA to a food base, such as skimmed milk or milk or a milk-based composition and fermentation as known.

In some embodiments, the composition is a drink that can be a functional drink or a therapeutic drink, a thirst-quencher or an ordinary drink. By way of example, the composition of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, acidified soy/juice beverage, aseptic/retorted chocolate drink, bar mixes, beverage powders, calcium fortified soy/plain and chocolate milk, calcium fortified coffee beverage.

In some embodiments, the SCFA is administered to the subject in a form of a pharmaceutical composition. For instance, the SCFA may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The SCFA can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

As used herein, the term “therapeutically effective amount” is an equivalent phrase refers to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease.

A further object of the present invention relates to a method for determining whether a subject suffering from an influenza infection is at risk of having a bacterial superinfection comprising i) determining the amount of short-chain fatty acid in a sample obtained from the subject, ii) comparing the amount determined at step i) with a predetermined reference value and iii) concluding that the subject is at risk of having a bacterial superinfection when the amount determined at step i) is lower than the predetermined reference value.

As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(l−p) where p is the probability of event and (1−p) is the probability of no event) to no—conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal.

In some embodiments, the sample is a fecal sample or a blood sample. In some embodiments, the blood sample is a serum sample or a plasma sample.

Methods for quantifying SCFAs in a sample are well known in the art. Typically, chemical derivitization and analysis by GC/MS (i.e. gas chromatography coupled to mass spectrometry) is a technique that is commonly used for characterization of SCFA (see e.g. Zhao G, Liu J F, Nyman M, Jonsson J A. Determination of short-chain fatty acids in serum by hollow fiber supported liquid membrane extraction coupled with gas chromatography. J Chromatogr B Analyt Technol Biomed Life Sci. 2007;846(1-2):202-208.). Liquid chromatography coupled to mass spectrometry is also suitable (see. WO 2010105112).

In some embodiments, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of level of SCFAs in properly banked historical patient samples may be used in establishing the predetermined reference value. In some embodiments, the predetermined reference value is derived from the level of SCFA in a control sample derived from one or more subjects who are substantially healthy (i.e. a normal BMI as above defined). The predetermined reference value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the level of the marker in a group of reference, one can use algorithmic analysis for the statistic treatment of the measured levels of the marker in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, when it is concluded that the subject is a risk of having a bacterial superinfection, then he can be administered with a therapeutically effective amount of SCFA as described above.

The invention will be further illustrated by the following figures and examples. However, these examples and figures should not be interpreted in any way as limiting the scope of the present invention.

### FIGURES

FIG. 1. Protective effect of acetate treatment on the control of pneumococcal infection. (A) Mice were treated or not with acetate five days before with S. pneumoniae (1×10⁶ CFUs) challenge. The number of bacteria was determined in lungs (upper panel) and spleen (lower panel) 30 hours after S. pneumoniae challenge. The solid lines correspond to the median values. A pool of two experiments is shown (n=18). (B) The mean number of bacteria+/−SD was determined in acetate-treated wild type (WT) and Ffar2^{-/-} mice. A representative experiment is shown (n=5). (A, B), **P<0.01, *P<0.05 (in a Mann-Whitney U test).

FIG. 2. Reduced metabolic activity of the gut microbiota during influenza infection. (A), Cecal concentrations of total SCFAs in mock-infected mice and 7 and 14 days post-IAV (H3N2) infection (left panel). The concentration of the main SCFAs in gut microbiota is depicted in the right panel (n=24, three pooled experiments). (B), Cecal concentrations of total SCFAs during the course of IAV (H1N1) infection (n=8). Data are expressed as mean±SD. Significant differences were determined using a Kruskal-Wallis one-way ANOVA test (A) or a Mann-Whitney U test (B) (***, P<0.001).

FIG. 3. Enhanced susceptibility to respiratory bacterial infection of mice colonized with IAV-experienced microbiota. The microbiota from mock-infected (control) mice or from IAV-infected (7 dpi) mice (A, H3N2, B, H1N1) was transplanted to ABX-treated mice. Three days later, colonized mice were challenged with S. pneumoniae (1×10⁶ CFUs). Bacterial loads were determined 30 hrs later. The solid lines correspond to the median values. A pool of two experiments is shown. *P<0.05, **, P<0.01) (in a Mann-Whitney U test).

FIG. 4. Protective effect of acetate treatment on the control of bacterial infection in colonized (IAV-experienced microbiota) mice and in IAV-infected mice. (A) The microbiota from IAV-infected (7 dpi, H1N1) mice was transplanted to ABX-treated mice and three days later, colonized mice were challenged with S. pneumoniae (1×10⁶ CFUs). Colonized mice were treated or not with acetate five days before S. pneumoniae challenge. (B) IAV (H1N1, WSN/33)-infected mice were treated with acetate at day 2 post-infection and were challenged with S. pneumoniae (1×10³ CFUs) at 7 dpi. (A and B), CFUS were determined (lungs and spleen) 30 hrs later. The solid lines correspond to the median values. A pool of two experiments is shown. *P<0.05, **, P<0.01 (in a Mann-Whitney U test).

FIG. 5. Protective effect of acetate treatment on the control of bacterial infection in IAV-infected mice. IAV (H1N1, A/California/04/2009)-infected mice were treated with acetate at day 2 post-infection and were challenged with S. pneumoniae (1×10³ CFUs) at 7 dpi. (A), CFUS were determined in the lungs (upper panel) and in the spleen (lower panel) 30 hrs later. The solid lines correspond to the median values. A representative experiment out of two is shown (n=8-9) (Mann-Whitney U test). (B). The survival of superinfected animals was monitored (n=14/group, two pooled experiments) (Kaplan-Meier analysis and log-rank test). *P<0.05, **, P<0.01.

### EXAMPLE

Methods

Mice, Ethics Statement and Reagents

Specific pathogen-free C57BL/6 mice (6-8 week-old, male) were purchased from Janvier (Le Genest-St-Isle, France). Mice were maintained in a biosafety level 2 facility in the Animal Resource Center at the Lille Pasteur Institute. All animal work conformed to the Lille Pasteur Institute animal care and used ethical guidelines (agreement number AF 16/20090 and 00357.03). Antibiotics were from Sigma-Aldrich (St Louis, Mo.) or R&D systems (Minneapolis, Minn.). SCFAs were from Sigma-Aldrich. FFAR2^{-/-} were described in (Maslowski et al., 2009).

Infections and Assessment of Bacterial Load

Mice were intranasally (i.n., 50μ1) infected with the high-pathogenicity mouse-adapted H3N2 IAV strain Scotland/20/74 (30 plaque forming units, PFUs), H1N1 IAV strain WSN/33 (200 PFUs) or H1N1 IAV strain A/California/04/2009) (100 PFUs) (Barthelemy et al., 2016; Barthelemy et al., 2017, Barthelemy et al. 2018). In the case of single bacterial infection, mice were i.n. inoculated with 1×10⁶ colony-forming units (CFUs) of S. pneumoniae serotype 1, a serotype linked to invasive pneumococcal disease (clinical isolate E1586). Superinfection was as follows. Mice were infected with IAV (the sub-lethale doses indicated above) and seven days later, animals were i.n. inoculated with 1×10³ CFUs of S. pneumoniae). A high severity model (prior infection with IAV H1N1, A/WSN/1933) and a mild severity model (prior infection with IAV H1N1, A/California/04/2009) of superinfection were used in this study. Enumeration of viable bacteria in lungs and spleen was determined 30 hours after the S. pneumoniae challenge. Survival was monitored daily after IAV infection and mice were euthanized when they lost in excess of 20% of their initial body weight.

Measurement of SCFA Concentrations and SCFA Treatment.

Concentrations of SCFAs in the cecal contents were determined using high-performance liquid chromatography by the internal standard method (LC-6A; Shimadzu, Kyoto, Japan) equipped with a Shim-pack SCR-102H column (inner diameter, 8 mm; length, 30 cm; Shimadzu) and a CDD-6A electroconductivity detector (Shimadzu). Naïve (non-infected) mice and IAV-infected mice were treated with acetate (200 mM, drinking water) five days before S. pneumoniae challenge (1×10⁶ CFUs and 1×10³ CFUs, respectively).

Fecal Transfer Experiments

Mice received broad-spectrum (fresh) antibiotics (ampicillin 2 g/L; neomycin 2 g/L, metronidazole 1 g/L, cyproflaxyn 1 g/L, nystatin 0.08 g/L and vancomycin 0.5 g/L) in drinking water for three weeks. The cages were changed every two days. Depletion of bacteria in the feces were checked by plating experiments. Antibiotic-treated mice were colonized (three days after antibiotics cessation) with 1×10⁹ bacteria recovered from mock-infected mice or from IAV-infected mice (7 dpi). The procedure was repeated two days after. One day after the last colonization, mice were infected with S. pneumoniae (1×10⁶ CFUs).

Statistical Analyses

A Mann-Whitney U test was used to compare two groups unless otherwise specified. Comparisons of more than two groups with each other were analyzed with the One-way Anova Kruskal-Wallis test (nonparametric), followed by the Dunn's posttest (PRISM v6 software, GraphPad Results are expressed as the mean±standard deviation (SD) unless otherwise stated. Survival of mice was compared using Kaplan-Meier analysis and log-rank test. A value of P<0.05 was considered as significant.

Results

Treatment with Acetate Protects Against Pneumococcal Infection

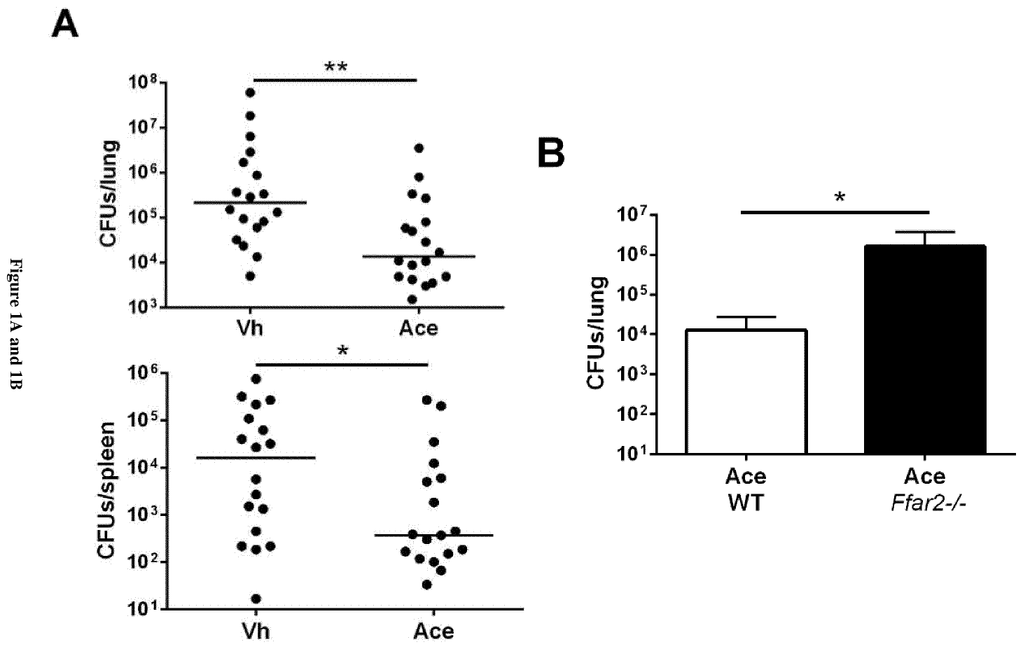

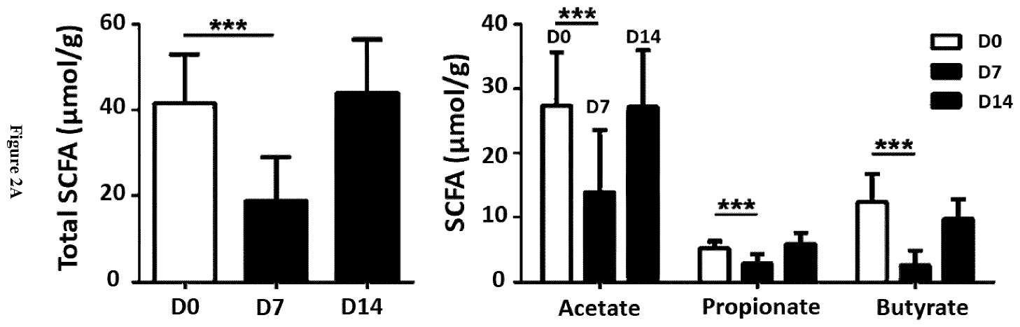

Short chain fatty acids are amongst the most abundant molecules produced by the gut bacteria. Production of gut microbiota-derived SCFAs has recently been shown to modulate pulmonary immune responses (asthma reaction), although the consequences on respiratory infections is still elusive (Maslowsky et al., 2009; Thorburn et al., 2015; Trompette et al., 2014; Cait et al., 2017). To investigate the effect of SCFAs on host defense against S. pneumoniae, mice were treated during five days with acetate (drinking water), the major specie of SCFAs. Compared to vehicle-treated animals, acetate supplementation significantly reduced the number of bacteria in the lungs (FIG. 1A, upper panel). Streptococcus pneumoniae can disseminate out of the lungs to become invasive. Of interest, acetate supplementation also lowered the number of bacteria in the spleen (FIG. 1A, lower panel). Hence, acetate boosts lung immunity to control pneumococcal infection. Acetate predominantly acts through the G-coupled receptor FFAR2 (also known as GPR43) and, to a lower extent (at least in humans), to FFA3 (GPR41) (Milligan G et al., 2017). As depicted in Figure. 1B, and relative to FFA2-competent mice, the protective effect of acetate was significantly reduced in Ffa2^{-/-} mice.

Influenza Infection Alters the Fermentation Activity of the Gut Microbiota

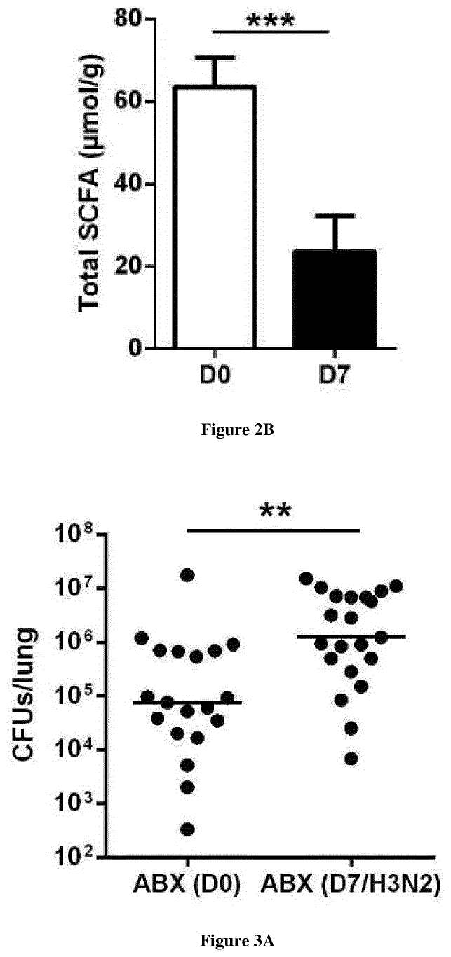

We next questioned whether a drop of SCFA production during severe influenza could lower pulmonary host defense against pneumococcal infection. Before this, we measured SCFA production in the context of influenza infection. As shown in FIG. 2A (left panel), the concentration of total SCFAs was significantly diminished 7 days post-influenza (H3N2) infection to return to basal level at 14 dpi. The concentration of acetate (C2), propionate (C3) and butyrate (C4), was significantly decreased at 7 dpi (FIG. 2A, right panel). Altered production of SCFAs was also observed in mice infected with H1N1 IAV (FIG. 2B and not shown). Together, this suggests that severe influenza alters the metabolic activity of the gut microbiota.

IAV-Experienced Microbiota Confers Susceptibility to Respiratory Bacterial Infection

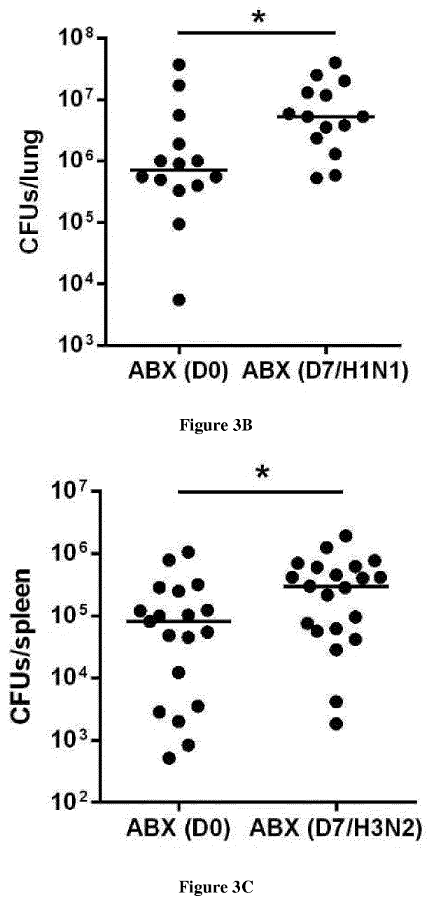

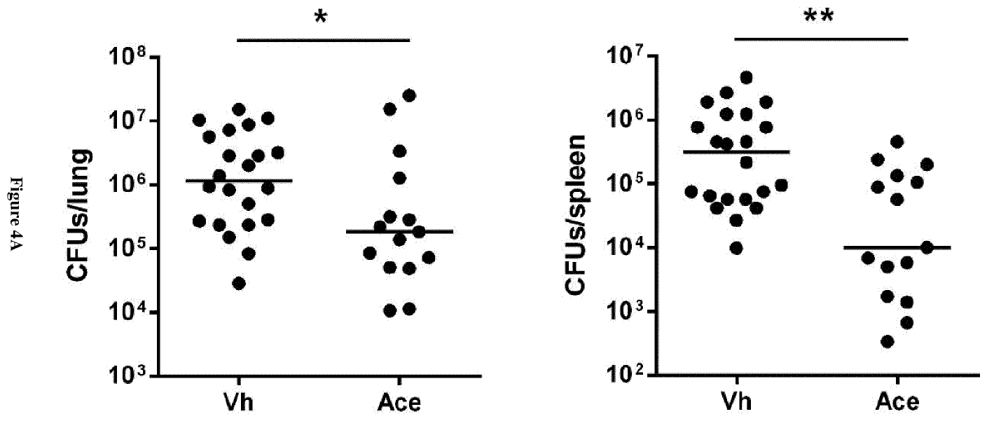

We next investigated whether IAV-experienced microbiota (through SCFA production) could confer susceptibility to respiratory bacterial infection. To this end, fecal transfer experiments were performed in mice previously treated with antibiotics, a procedure that transiently depletes host commensals. The microbiota collected from mock-infected (control) mice and from IAV (H3N2)-infected mice (7 dpi) was transplanted to ABX-treated mice. Remarkably, and compared to the control group, the transfer of IAV-experienced microbiota enhanced susceptibility to pneumococcal infection (FIG. 3A). To investigate whether this is also the case during H1N1 infection, the same procedure was repeated but this time with the microbiota collected from IAV (H1N1)-infected mice. As depicted in FIG. 3B, mice colonized with IAV-experienced microbiota had a higher number of bacteria in the lungs. In both systems (H3N2 and H1N1), IAV microbiota also enhanced bacterial dissemination out of the lungs (FIG. 3C and not shown). These data suggest that disruption of the intestinal bacterial homeostasis during influenza infection is sufficient to confer susceptibility to respiratory bacterial infection.

Reduced SCFA Production by IAV-Experienced Microbiota Enhances Secondary Bacterial Infection

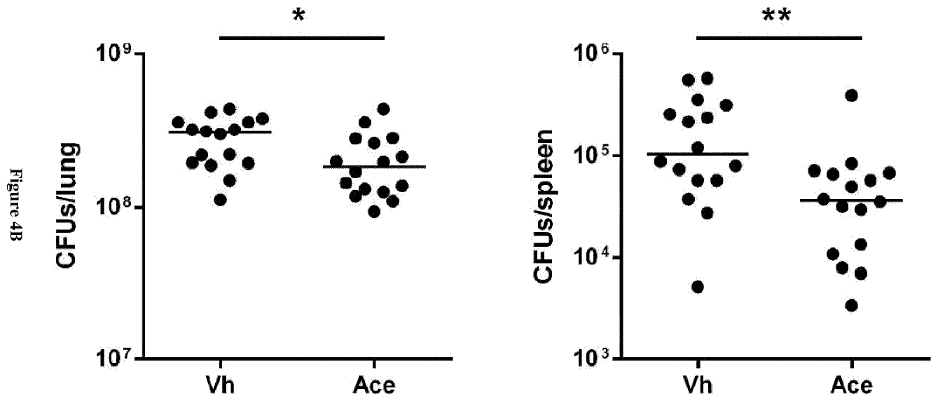

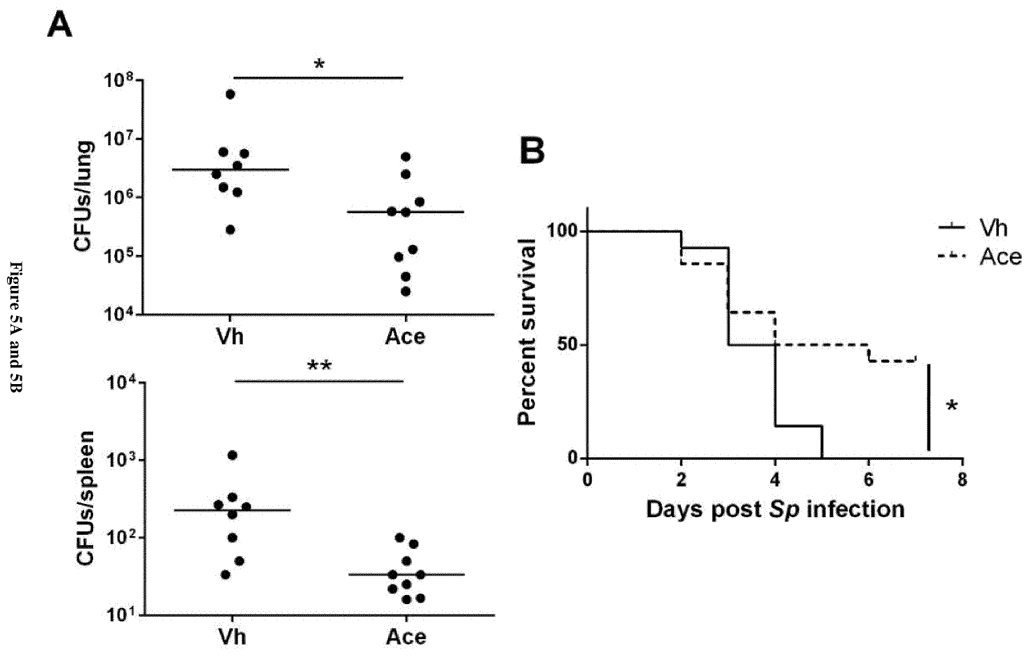

We next questioned whether acetate could rescue the altered response in colonized (acetate-deficient) mice. Remarkably, mice colonized with IAV microbiota and treated with acetate were more resistant to S. pneumoniae challenge compared to vehicle-treated transplanted animals (FIG. 4A). Indeed acetate-treated mice had a lower number of pneumococci in lungs. Acetate treatment also lowered the systemic dissemination of pneumococci out of the lungs. These data suggested that defective SCFA (acetate) production during severe influenza might enhance secondary bacterial infection. To further demonstrate this, IAV-infected mice were treated with acetate before the secondary bacterial challenge. To this end, a severe (H3N2) and a milder (H1N1p) models were developed. In IAV (H3N2)-infected mice supplemented with acetate (at day 2 p.i.), a significant lower bacterial load in the lung and spleen was observed (FIG. 4B). The effect of acetate in the less severe superinfection model was next assessed. Acetate treatment significantly reduced the bacterial loads in the lung and spleen (FIG. 5A). Lastly, and importantly, acetate treatment resulted in a significant enhancement of the survival rate of double-infected mice (FIG. 5B). Taken as a whole, low SCFA production by the gut microbiota during influenza infection influences susceptibility to secondary bacterial infection and restoration of acetate is sufficient to improve disease outcomes.

Discussion:

The present study aimed at analyzing the impact of severe influenza on the functionality (fermentation activity) of the gut microbiota and at studying the impact of potential alterations on secondary respiratory bacterial infection, a phenomenon that arises following local immune suppression. This study demonstrates the impact of microbiota alterations during influenza infection on secondary bacterial infection. It also highlights the importance of gut microbiota-derived SCFAs in pulmonary innate immunity against bacterial infections, including in the context of prior influenza.

Emerging evidences suggest that natural (humans, H7N9) and experimental (mouse, H1N1) influenza infection alters the composition of the gut microbiota (Wang et al., 2014; Lu e al., 2014; Qin et al., 2015; Deriu et al., 2016; Bartley et al., 2017). We have recently analyzed, on a large number of mice, the impact of severe H3N2 and H1N1 influenza—the two mains subtypes in humans-on the diversity and composition of the gut (caecal and colonic) microbiota. We have confirmed that severe influenza alter the relative abundances of microbial taxa at 7 dpi (manuscript in preparation). In contrast to chronic pathologies where a strong decrease of phylogenic diversity arises (dysbiosis), marked changes occurred on phylogenetic specifications, with no major decreased global diversity. We did uncover several shared responses of the microbiota between H3N2 and H1N1. In the current study, we show for the first time that alteration of the gut microbiota composition at 7 dpi is associated with a concurrent drop in the concentration of intestinal SCFAs, an effect probably due to reduced frequencies of bacterial SCFA producers. Among candidates (reduced frequencies at 7 dpi) are S24-7 family (Bacteroidetes) and Lachnospiraceae genus (Firmicutes) which are notable for containing many species capable of fermenting complex carbohydrates to SCFAs (Louis et Flint, 2017; Vital et al., 2014).

What are the consequences of altered microbiota on disease outcomes during influenza? Deriu and collaborators elegantly demonstrated that gut disorders during influenza favors local colonization and systemic dissemination of Salmonella Typhimurium, a leading cause of acute gastroenteritis (Deriu et al., 2016). The mechanisms leading to this enhanced secondary enteric susceptibility are still elusive and might result from a relaxed intestinal barrier and/or local immune suppression. Whether the drop of SCFA production during influenza plays a role in this setting is an open question. Whatever the mechanisms, this datum suggests that secondary (systemic) infection during severe influenza might originate from the gut compartment, and not solely from the pulmonary compartment. Whether altered gut microbiota might predispose to secondary pulmonary bacterial infection was addressed for the first time in the current study. This is a particularly significant question in view of reported cases of fatal respiratory superinfection during severe influenza. In the present study, we demonstrated that gut microbiota (metabolic) changes have implication in secondary respiratory bacterial infection post-influenza. Indeed, transfer experiments indicate that the altered microbiota affects the pulmonary host response to S. pneumoniae. Acetate is the likely mediator of this defective host response as acetate (the main SCFA distributed throughout the body) in drinking water rescued host defense mechanisms in our settings. To the best of our knowledge, this is the first time that altered SCFA production during a stressful condition leads to impaired immunity and secondary infection.

The role of acetate (and more generally bacterial metabolites such as SCFAs) in pulmonary host defenses is largely elusive. Short-chain fatty acids are derived from the anaerobic fermentation of non-digestible polysaccharides, such as resistant starches and dietary fibers. Microbial-derived SCFAs exert many physiologic functions in the intestine where their concentration in the gut lumen can reach 100-200 mM (Thorburn et al., 2014). They are (primarily butyrate) a source for host colonic epithelium and enhance intestinal barrier properties. SCFAs display anti-oxidative and anti-inflammatory functions, thus protecting against tumor growth and colitis (Maslowski et al., 2009; Kim et al., 2013). Production of SCFAs is important in the control of the gut microbiota as they can act as energy source for certain bacterial species. Intestinal SCFAs can also protect against enteric infection including shigellosis and salmonellosis (Rabbani et al., 1999; Raqib et al., 2006; Canani et al., 2011; Raqui et al., 2012; Sunkara et al., 2012). SCFAs can diffuse in the blood (0.1-1 mM) and activate numerous physiologic processes. They are very important in the so-called “gut-brain” axis and in the stimulation of neural and hormonal signals regulating energy homeostasis (Kuwahara, 2014). Recent evidences suggest that SCFAs can act in the lung compartment to modulate pulmonary immunity. For instance, Trompette and coauthors showed that propionate, by acting on dendritic cell progenitors in the bone marrow, lowers Th2 response to allergens, thus decreasing asthma reactions (Trompette et al., 2014). On the other hand, butyrate supplementation alters the function of systemic dendritic cells to ameliorate asthma (Cait et al., 2017). More recently, it was shown that butyrate controls influenza infection by reducing, through enhanced CD8⁺ T cell activity, the viral replication (Trompette et al. 2018). SCFAs act though G-protein coupled receptors and/or through HDAC inhibition. The potential function (through supplementation) of SCFAs in respiratory bacterial infections has recently been reported. Interestingly, combined butyrate and vitamin D3 treatment ameliorates pulmonary tuberculosis in humans, probably by boosting antibacterial functions of macrophages (Milₜ′ et al., 2015). On the other hand, Ciarlo and coauthors show that propionate failed to modulate host defenses against respiratory bacterial (including pneumococcal) and fungal infections (Ciarlo et al., 2016). Our data contrast with this later study, a finding that can be explained by the use of acetate in the current study and the different protocols used for infection. Our data suggests that the protective effect of acetate during S. pneumoniae infection depends on FFAR2 expression. Of importance, treatment of IAV-infected mice with acetate results in a better control of bacterial growth in the lungs and dissemination out of the lungs (both in the severe and mild models of surinfection). In the later system, acetate treatment also resulted in enhanced survival rate. Immunological mechanisms sustaining these protective effects are still elusive and may be due to enhanced number and/or functions of effector cells (macrophages, neutrophils) in the lungs. Cells involved in their activation, including dendritic cells and non-conventional T cells (γδ and NKT cells for instance) may also be impacted. Whatever the mechanisms, our data highlight the positive role of SCFA supplementation (used in the clinics) during influenza. This finding might have therapeutic applications in many settings including in acute diseases associated with altered microbiota and secondary infections such as trauma, burn and sepsis. To conclude, we propose that altered gut microbiota during acute infection (influenza) acts as a critical factor in secondary respiratory bacterial infection through suppression of host pulmonary immunity. Our findings might have therapeutic applications (SCFAs or FFAR agonists) in diseases associated with dysbiosis and secondary bacterial infections.

### REFERENCES

Throughout this application, various references describe the state of the art to which this invention pertains. The disclosures of these references are hereby incorporated by reference into the present disclosure.
Arpaia N, Campbell C, Fan X, Dikiy S, van der Veeken J, deRoos P, et al. Metabolites produced by commensal bacteria promote peripheral regulatory T-cell generation (2013). Nature. 504:451-5.
Ballinger M N, Standiford T J. Postinfluenza bacterial pneumonia: host defenses gone awry (2010). J Interferon Cytokine Res. 30:643-52.
Barthelemy A, Ivanov S, Fontaine J, Soulard D, Bouabe H, Paget C, et al. Influenza A virus-induced release of interleukin-10 inhibits the anti-microbial activities of invariant natural killer T cells during invasive pneumococcal superinfection (2017). Mucosal Immunol. 10:460-9.
Barthelemy A, Ivanov S, Hassane M, Fontaine J, Heurtault B, Frisch B, et al. Exogenous Activation of Invariant Natural Killer T Cells by α-Galactosylceramide Reduces Pneumococcal Outgrowth and Dissemination Postinfluenza (2016). MBio. 7. pii: e01440-16
Barthelemy, A., Sencio, V., Soulard, V., Deruytter, L., Le Goffic, R and Trottein, F. Interleukin-22 immunotherapy modulates pulmonary gene expression evocative of enhanced tissue integrity and reduces bacterial systemic invasion during severe influenza. Infection & Immunity (2018). 86:717.
Bartley J M, Zhou X, Kuchel G A, Weinstock G M, Haynes L. Impact of Age, Caloric Restriction, and Influenza Infection on Mouse Gut Microbiome: An Exploratory Study of the Role of Age-Related Microbiome Changes on Influenza Responses (2017). Front Immunol. 8:1164.
Belkaid Y, Hand T W. Role of the microbiota in immunity and inflammation (2014). Cell. 157:121-41.
Brundage J F. Interactions between influenza and bacterial respiratory pathogens: implications for pandemic preparedness (2006). Lancet Infect Dis. 6:303-12.
Cait A, Hughes M R, Antignano F, Cait J, Dimitriu P A, Maas K R, et al. Microbiome-driven allergic lung inflammation is ameliorated by short-chain fatty acids (2017). Mucosal Immunol.
Canani R B, Costanzo M D, Leone L, Pedata M, Meli R, Calignano A. Potential beneficial effects of butyrate in intestinal and extraintestinal diseases (2011). World J Gastroenterol. 17:1519-28.
Ciarlo E, Heinonen T, Herderschee J, Fenwick C, Mombelli M, Le Roy D, et al. Impact of the microbial derived short chain fatty acid propionate on host susceptibility to bacterial and fungal infections in vivo (2016). Sci Rep. 6:37944.
Clarke T B. Microbial Programming of Systemic Innate Immunity and Resistance to Infection (2014). PLOS Pathogens. 10:e1004506.
Clemente J C, Ursell L K, Parfrey L W, Knight R. The impact of the gut microbiota on human health: an integrative view (2012). Cell. 148:1258-70.
Deriu E, Boxx G M, He X, Pan C, Benavidez S D, Cen L, et al. Influenza Virus Affects Intestinal Microbiota and Secondary Salmonella Infection in the Gut through Type I Interferons (2016). PLoS Pathog. 12:e1005572.
Gray J, Oehrle K, Worthen G, Alenghat T, Whitsett J, Deshmukh H. Intestinal commensal bacteria mediate lung mucosal immunity and promote resistance of newborn mice to infection. Sci Transl Med (2017). 9.
Hooper L V, Littman D R, Macpherson A J. Interactions between the microbiota and the immune system (2012). Science. 336:1268-73.
Huffnagle G B. Increase in dietary fiber dampens allergic responses in the lung (2014). Nat Med. 20:120-1.
Kim M H, Kang S G, Park J H, Yanagisawa M, Kim C H. Short-chain fatty acids activate GPR41 and GPR43 on intestinal epithelial cells to promote inflammatory responses in mice (2013). Gastroenterology. 145:396-406-10.
Kuwahara A. Contributions of colonic short-chain Fatty Acid receptors in energy homeostasis (2014). Front Endocrinol (Lausanne). 5:144.
Levy M, Kolodziejczyk A A, Thaiss C A, Elinav E. Dysbiosis and the immune system (2017). Nat Rev Immunol. 17:219-32.
Louis P, Flint H J. Diversity, metabolism and microbial ecology of butyrate-producing bacteria from the human large intestine (2009). FEMS Microbiol Lett. 294:1-8.
Lu H, Zhang C, Qian G, Hu X, Zhang H, Chen C, et al. An analysis of microbiota-targeted therapies in patients with avian influenza virus subtype H7N9 infection (2014). BMC Infect Dis. 14:359.
Macfarlane S, Macfarlane G T. Regulation of short-chain fatty acid production (2003). Proc Nutr Soc. 62:67-72.
Macia L, Tan J, Vieira A T, Leach K, Stanley D, Luong S, et al. Metabolite-sensing receptors GPR43 and GPR109A facilitate dietary fibre-induced gut homeostasis through regulation of the inflammasome (2015). Nat Commun. 1; 6:6734.
Marsland B J, Gollwitzer E S. Host-microorganism interactions in lung diseases (2014). Nat Rev Immunol. 14:827-35.
Maslowski K M, Vieira A T, Ng A, Kranich J, Sierro F, Yu D, et al. Regulation of inflammatory responses by gut microbiota and chemoattractant receptor GPR43 (2009). Nature. 461:1282-6.
McCullers J A. Insights into the Interaction between Influenza Virus and Pneumococcus (2006). Clin Microbiol Rev. 19:571-82.
Mily A, Rekha R S, Kamal S M M, Arifuzzaman A S M, Rahim Z, Khan L, et al. Significant Effects of Oral Phenylbutyrate and Vitamin D3 Adjunctive Therapy in Pulmonary Tuberculosis: A Randomized Controlled Trial (2015). PLoS ONE. 10e0138340.
Milligan G, Bharat S, Ulven T and Hudson B D (2017). Complex Pharmacology of Free Fatty Acid Receptors. Chem Rev. 117: 67-110.
Monto A S. Epidemiology of influenza (2008). Vaccine. 26 Suppl 4:D45-48.
Morens D M, Taubenberger J K, Fauci A S. Predominant role of bacterial pneumonia as a cause of death in pandemic influenza: implications for pandemic influenza preparedness (2008). J Infect Dis. 198:962-70.
Qin N, Zheng B, Yao J, Guo L, Zuo J, Wu L, et al. Influence of H7N9 virus infection and associated treatment on human gut microbiota (2015). Sci Rep. 5:14771.
Rabbani G H, Albert M J, Hamidur Rahman A S, Moyenul Isalm M, Nasirul Islam K M, Alam K. Short-chain fatty acids improve clinical, pathologic, and microbiologic features of experimental shigellosis (199). J Infect Dis. 179:390-7.
Raqib R, Sarker P, Bergman P, Ara G, Lindh M, Sack D A, et al. Improved outcome in shigellosis associated with butyrate induction of an endogenous peptide antibiotic (2006). Proc Natl Acad Sci USA. 103:9178-83.
Raqib R, Sarker P, Mily A, Alam N H, Arifuzzaman A S M, Rekha R S, et al. Efficacy of sodium butyrate adjunct therapy in shigellosis: a randomized, double-blind, placebo-controlled clinical trial (2012). BMC Infect Dis. 12:111.
Schuijt T J, Lankelma J M, Scicluna B P, de Sousa E Melo F, Roelofs J J T H, de Boer J D, et al. The gut microbiota plays a protective role in the host defence against pneumococcal pneumonia (2016). Gut. 65:575-83.
Short K R, Kroeze E J B V, Fouchier R A M, Kuiken T. Pathogenesis of influenza-induced acute respiratory distress syndrome (2014). Lancet Infect Dis. 14:57-69.
Smith P M, Howitt M R, Panikov N, Michaud M, Gallini C A, Bohlooly-Y M, et al. The microbial metabolites, short-chain fatty acids, regulate colonic Treg cell homeostasis (2013). Science. 341:569-73.
Snelgrove R J, Godlee A, Hussell T. Airway immune homeostasis and implications for influenza-induced inflammation (2011). Trends Immunol. 32:328-34.
Sunkara L T, Jiang W, Zhang G. Modulation of antimicrobial host defense peptide gene expression by free fatty acids (2012). PLoS ONE. 7:e49558.
Tan J, McKenzie C, Potamitis M, Thorburn A N, Mackay C R, Macia L. The role of short-chain fatty acids in health and disease (2014). Adv Immunol. 121:91-119.
Tan J, McKenzie C, Vuillermin P J, Goverse G, Vinuesa C G, Mebius R E, et al. Dietary Fiber and Bacterial SCFA Enhance Oral Tolerance and Protect against Food Allergy through Diverse Cellular Pathways (2016). Cell Rep. 15:2809-24.
Taubenberger J K, Morens D M. The pathology of influenza virus infections (2008). Annu Rev Pathol. 3:499-522.
Thaiss C A, Zmora N, Levy M, Elinav E. The microbiome and innate immunity (2016). Nature. 535:65-74.
Thorburn A N, Macia L, Mackay C R. Diet, metabolites, and «western-lifestyle» inflammatory diseases (2014). Immunity. 40:833-42.
Thorburn A N, McKenzie C I, Shen S, Stanley D, Macia L, Mason L J, et al. Evidence that asthma is a developmental origin disease influenced by maternal diet and bacterial metabolites (2015). Nat 6:7320.
Topping D L, Clifton P M. Short-chain fatty acids and human colonic function: roles of resistant starch and nonstarch polysaccharides (2001). Physiol Rev. 81:1031-64.
Trompette A, Gollwitzer E S, Yadava K, Sichelstiel A K, Sprenger N, Ngom-Bru C, et al. Gut microbiota metabolism of dietary fiber influences allergic airway disease and hematopoiesis (2014). Nat Med. 20:159-66.
Trompette A, Gollwitzer E S, Pattaroni C, Lopez-Mejia I C, Riva E, Pernot J, Ubags N, Fajas L, Nicod L P, Marsland B J. Dietary Fiber Confers Protection against Flu by Shaping Ly6c-Patrolling Monocyte Hematopoiesis and CD8+ T Cell Metabolism. Immunity 48: 992-1005.
van der Sluijs K F, van der Poll T, Lutter R, Juffermans N P, Schultz M J. Bench-to-bedside review: bacterial pneumonia with influenza—pathogenesis and clinical implications (2010). Crit Care. 14:219.
Vital M, Howe A C, Tiedje J M. Revealing the bacterial butyrate synthesis pathways by analyzing (meta)genomic data (2014). MBio. 5:e00889.
Wang J, Li F, Wei H, Lian Z-X, Sun R, Tian Z. Respiratory influenza virus infection induces intestinal immune injury via microbiota-mediated Th17 cell-dependent inflammation (214). J Exp Med. 211:2397-410.
Wong J M W, de Souza R, Kendall C W C, Emam A, Jenkins D J A. Colonic health: fermentation and short chain fatty acids (2006). J Clin Gastroenterol. 40:235-43.
Yang J, Martinez I, Walter J, Keshavarzian A, Rose D J. In vitro characterization of the impact of selected dietary fibers on fecal microbiota composition and short chain fatty acid production (2013). Anaerobe. 23:74-81.

1. A method of treating a bacterial superinfection post-influenza in a subject in need thereof comprising administering to the subject a therapeutically effective amount of at least one short-chain fatty acid (SCFA) after an influenza infection.

2. The method of claim 1 wherein the at least one SCFA is selected from free fatty acid receptor 2 (FFAR2) agonist or free fatty acid receptor 3 (FFAR3) agonist.

3. The method of claim 1 wherein the influenza infection is caused by Influenza virus A or B.

4. The method of claim 3 wherein the influenza virus A is H1N1, H2N2, H3N2 or H5N1.

5. The method of claim 1 wherein the bacterial superinfection is selected from the group consisting of lower respiratory tract infections, middle ear infections and bacterial sinusitis.

6. The method of claim 1 wherein the bacterial superinfection is caused by at least one organism selected from the group consisting of Streptococcus pneumoniae; Staphylococcus aureus; Haemophilus influenza, Myoplasma species and Moraxella catarrhalis.

7. The method of claim 1 wherein the subject is selected from the group consisting of subjects who are at least 50 years old, subjects who reside in chronic care facilities, subjects who have chronic disorders of the pulmonary or cardiovascular system, subjects who required regular medical follow-up or hospitalization during the preceding year because of chronic metabolic diseases, renal dysfunction, hemoglobinopathies, or immunosuppression, children less than 14 years of age, patients between 6 months and 18 years of age who are receiving long-term aspirin therapy, and women who will be in the second or third trimester of pregnancy during the influenza season.

8. The method of claim 1 wherein the subject is older than 1 year old and less than 14 years old; between the ages of 50 and 65, or older than 65 years of age.

9. The method of claim 1 wherein the at least one SCFA is selected from saturated fatty acids comprising six or less carbon atoms, or 5 or less carbon atoms.

10. The method of claim 1 wherein the at least one SCFA is selected from the group consisting of formic acid, acetic acid, propionic acid, butyric acid, isobutyric acid, valeric acid, isovaleric acid, caproic acid, formate, acetate, butyrate, isobutyrate, valerate, isovalerate, caproate, propionic acid and propionate.

11. The method of claim 1 wherein the SCFA is administered to the subject in the form of a nutritional composition.

12. The method of claim 1 wherein the SCFA is administered to the subject in the form of a food composition.

13. The method of claim 1 wherein the SCFA is administered to the subject in a pharmaceutical composition.

14. A method for determining whether a subject suffering from an influenza infection is at risk of having a bacterial superinfection and treating the subject, comprising i) determining the amount of short-chain fatty acid in a sample obtained from the subject, ii) comparing the amount determined at step i) with a predetermined reference value and iii) administering a at least one short chain fatty acid to the subject when the amount determined at step i) is lower than the predetermined reference value.

15. The method of claim 14 wherein the sample is a fecal sample or a blood sample.
